(19) United States
(12) Patent Application Publication
(10) Pub. No.: US 2007/0219431 A1
(43) Pub. Date: Sep. 20, 2007

(54) Method to Facilitate Providing Access to a Plurality of Private Civil Security Resources

(76) Inventor: Barrett H. Moore, Winnetka, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/549,874

(22) Filed: Oct. 16, 2006

Related U.S. Application Data
(63) Continuation-in-part of application No. 11/384,037, filed on Mar. 17, 2006.
Application No. 11/384,037 is a continuation-in-part of application No. 11/394,350, filed on Mar. 30, 2006.
Application No. 11/394,350 is a continuation-in-part of application No. 11/279,333, filed on Apr. 11, 2006.
Application No. 11/279,333 is a continuation-in-part of application No. 11/379,929, filed on Apr. 24, 2006.
Application No. 11/379,929 is a continuation-in-part of application No. 11/381,247, filed on May 2, 2006.
Application No. 11/381,247 is a continuation-in-part of application No. 11/381,257, filed on May 2, 2006.
Application No. 11/381,257 is a continuation-in-part of application No. 11/381,265, filed on May 2, 2006.
Application No. 11/381,265 is a continuation-in-part of application No. 11/381,277, filed on May 2, 2006.
Application No. 11/381,277 is a continuation-in-part of application No. 11/383,022, filed on May 12, 2006.
Application No. 11/383,022 is a continuation-in-part of application No. 11/420,594, filed on May 26, 2006.
Application No. 11/420,594 is a continuation-in-part of application No. 11/421,694, filed on Jun. 1, 2006.
Application No. 11/421,694 is a continuation-in-part of application No. 11/423,594, filed on Jun. 12, 2006.
Application No. 11/423,594 is a continuation-in-part of application No. 11/425,043, filed on Jun. 19, 2006.
Application No. 11/425,043 is a continuation-in-part of application No. 11/426,231, filed on Jun. 23, 2006.
Application No. 11/426,231 is a continuation-in-part of application No. 11/456,472, filed on Jul. 10, 2006.
Application No. 11/456,472 is a continuation-in-part of application No. 11/461,605, filed on Aug. 1, 2006.
Application No. 11/461,605 is a continuation-in-part of application No. 11/461,624, filed on Aug. 1, 2006.
Application No. 11/461,624 is a continuation-in-part of application No. 11/462,795, filed on Aug. 7, 2006.
Application No. 11/462,795 is a continuation-in-part of application No. 11/462,845, filed on Aug. 7, 2006.
Application No. 11/462,845 is a continuation-in-part of application No. 11/464,751, filed on Aug. 15, 2006.
Application No. 11/464,751 is a continuation-in-part of application No. 11/464,764, filed on Aug. 15, 2006.
Application No. 11/464,764 is a continuation-in-part of application No. 11/464,775, filed on Aug. 15, 2006.
Application No. 11/464,775 is a continuation-in-part of application No. 11/464,788, filed on Aug. 15, 2006.
Application No. 11/464,788 is a continuation-in-part of application No. 11/464,799, filed on Aug. 15, 2006.
Application No. 11/464,799 is a continuation-in-part of application No. 11/465,063, filed on Aug. 16, 2006.
Application No. 11/465,063 is a continuation-in-part of application No. 11/466,727, filed on Aug. 23, 2006.
Application No. 11/466,727 is a continuation-in-part of application No. 11/466,953, filed on Aug. 24, 2006.
Application No. 11/466,953 is a continuation-in-part of application No. 11/470,156, filed on Sep. 5, 2006.
Application No. 11/470,156 is a continuation-in-part of application No. 11/531,651, filed on Sep. 13, 2006.
Application No. 11/531,651 is a continuation-in-part of application No. 11/532,461, filed on Sep. 15, 2006.
Application No. 11/532,461 is a continuation-in-part of application No. 11/535,021, filed on Sep. 25, 2006.
Application No. 11/535,021 is a continuation-in-part of application No. 11/535,282, filed on Sep. 26, 2006.
Application No. 11/535,282 is a continuation-in-part of application No. 11/537,469, filed on Sep. 29, 2006.
Application No. 11/537,469 is a continuation-in-part of application No. 11/539,798, filed on Oct. 9, 2006.
Application No. 11/539,798 is a continuation-in-part of application No. 11/539,861, filed on Oct. 9, 2006.
Application No. 11/539,861 is a continuation-in-part of application No. 11/548,191, filed on Oct. 10, 2006.
(60) Provisional application No. 60/820,628, filed on Jul. 28, 2006.
(60) Provisional application No. 60/823,806, filed on Aug. 29, 2006.
(60) Provisional application No. 60/825,524, filed on Sep. 13, 2006.
(60) Provisional application No. 60/825,976, filed on Sep. 18, 2006.
(60) Provisional application No. 60/826,491, filed on Sep. 21, 2006.
(60) Provisional application No. 60/827,591, filed on Sep. 29, 2006.
(60) Provisional application No. 60/827,820, filed on Oct. 2, 2006.
(60) Provisional application No. 60/827,828, filed on Oct. 2, 2006.
(60) Provisional application No. 60/828,341, filed on Oct. 5, 2006.
(60) Provisional application No. 60/828,342, filed on Oct. 5, 2006.
(60) Provisional application No. 60/829,447, filed on Oct. 13, 2006.

(51) Int. Cl. A61B 5/00

(52) U.S. Cl. 600/300

(57) ABSTRACT

A plurality of private civil security resources are provided (101). These private civil security resources comprise, at least in part, civil security provisions, civil security facilities, and civil security provisions. Authorized beneficiaries are then provided (103) with consideration-based access to this plurality of private civil security resources.

### RELATED APPLICATIONS

This application claims the benefit of the filing date of U.S. provisional application Nos. 60/820,628, 60/823,806, 60/825,524, 60/825,976, 60/826,491, 60/827,591, 60/827,820, 60/827,828, 60/828,341, 60/828,342, and 60/829,447 which are hereby incorporated in their entirety herein.

This comprises a continuation-in-part of each of:

SUBSCRIPTION-BASED PRIVATE CIVIL SECURITY FACILITATION METHOD as filed on Mar. 17, 2006 and having application Ser. No. 11/384,037;

SUBSCRIPTION-BASED CATASTROPHE-TRIGGERED MEDICAL SERVICES FACILITATION METHOD as filed on Mar. 30, 2006 and having application Ser. No. 11/394,350;

PERSONAL PROFILE-BASED PRIVATE CIVIL SECURITY SUBSCRIPTION METHOD as filed on Apr. 11, 2006 and having application Ser. No. 11/279,333;

RADIATION SHELTER KIT APPARATUS AND METHOD as filed on Apr. 24, 2006 and having application Ser. No. 11/379,929;

FRACTIONALLY-POSSESSED UNDERGROUND SHELTER METHOD AND APPARATUS as filed on May 2, 2006 and having application Ser. No. 11/381,247;

SUBSCRIPTION-BASED CATASTROPHE-TRIGGERED TRANSPORT SERVICES FACILITATION METHOD AND APPARATUS as filed on May 2, 2006 and having application Ser. No. 11/381,257;

SUBSCRIPTION-BASED MULTI-PERSON EMERGENCY SHELTER METHOD as filed on May 2, 2006 and having application Ser. No. 11/381,265;

SUBSCRIPTION-BASED CATASTROPHE-TRIGGERED RESCUE SERVICES FACILITATION METHOD AND APPARATUS as filed on May 2, 2006 and having application Ser. No. 11/381,277;

DOCUMENT-BASED CIVILLY-CATASTROPHIC EVENT PERSONAL ACTION GUIDE FACILITATION METHOD as filed on May 12, 2006 and having application Ser. No. 11/383,022;

RESCUE CONTAINER METHOD AND APPARATUS as filed on May 26, 2006 and having application Ser. No. 11/420,594;

PURCHASE OPTION-BASED EMERGENCY SUPPLIES PROVISIONING METHOD as filed on Jun. 1, 2006 and having application Ser. No. 11/421,694;

SUBSCRIPTION-BASED PRE-PROVISIONED TOWABLE UNIT FACILITATION METHOD as filed on Jun. 12, 2006 and having application Ser. No. 11/423,594;

RADIATION-BLOCKING BLADDER APPARATUS AND METHOD as filed on Jun. 19, 2006 and having application Ser. No. 11/425,043;

PRIVATE CIVIL DEFENSE-THEMED TELEVISION BROADCASTING METHOD as filed on Jun. 23, 2006 and having application Ser. No. 11/426,231;

EMERGENCY SUPPLIES PRE-POSITIONING AND ACCESS CONTROL METHOD as filed on Jul. 10, 2006 and having application Ser. No. 11/456,472;

PRIVATE CIVIL DEFENSE-THEMED BROADCASTING METHOD as filed on Aug. 1, 2006 and having application Ser. No. 11/461,605;

METHOD OF PROVIDING VARIABLE SUBSCRIPTION-BASED ACCESS TO AN EMERGENCY SHELTER as filed on Aug. 1, 2006 and having application Ser. No. 11/461,624;

SUBSCRIPTION-BASED INTERMEDIATE SHORT-TERM EMERGENCY SHELTER METHOD as filed on Aug. 7, 2006 and having application Ser. No. 11/462,795;

SUBSCRIPTION-BASED CATASTROPHE-TRIGGERED RESCUE SERVICES FACILITATION METHOD USING WIRELESS LOCATION INFORMATION as filed on Aug. 7, 2006 and having application Ser. No. 11/462,845;

PRIVATELY PROVISIONED SURVIVAL SUPPLIES DELIVERY METHOD as filed on Aug. 15, 2006 and having application Ser. No. 11/464,751;

PRIVATELY PROVISIONED SURVIVAL SUPPLIES SUB-UNIT-BASED DELIVERY METHOD as filed on Aug. 15, 2006 and having application Ser. No. 11/464,764;

PRIVATELY PROVISIONED SURVIVAL SUPPLIES ACQUISITION METHOD as filed on Aug. 15, 2006 and having application Ser. No. 11/464,775;

PRIVATELY PROVISIONED SURVIVAL SUPPLIES CONTENT ACQUISITION METHOD as filed on Aug. 15, 2006 and having application Ser. No. 11/464,788;

METHOD TO PRIVATELY PROVISION SURVIVAL SUPPLIES THAT INCLUDE THIRD PARTY ITEMS as filed on Aug. 15, 2006 and having application Ser. No. 11/464,799;

WASTE DISPOSAL DEVICE as filed on Aug. 16, 2006 and having application Ser. No. 11/465,063;

SUBSCRIPTION-BASED PRIVATE CIVIL SECURITY RESOURCE CUSTOMIZATION METHOD as filed on Aug. 23, 2006 and having application Ser. No. 11/466,727;

PREMIUM-BASED PRIVATE CIVIL SECURITY POLICY METHODS as filed on Aug. 24, 2006 and having application Ser. No. 11/466,953;

SUBSCRIPTION-BASED MOBILE SHELTER METHOD as filed on Sep. 5, 2006 and having application Ser. No. 11/470,156;

METHOD OF PROVIDING A FLOATING LIFE-SUSTAINING FACILITY as filed on Sep. 13, 2006 and having application Ser. No. 11/531,651;

PRIVATELY PROVISIONED SUB-UNIT-BASED SURVIVAL SUPPLIES PROVISIONING METHOD as filed on Sep. 15, 2006 and having application Ser. No. 11/532,461;

PRIVATELY PROVISIONED INTERLOCKING SUB-UNIT-BASED SURVIVAL SUPPLIES PROVISIONING METHOD as filed on Sep. 25, 2006 and having application Ser. No. 11/535,021;

RESOURCE CONTAINER AND POSITIONING METHOD AND APPARATUS as filed on Sep. 26, 2006 and having application Ser. No. 11/535,282;

PUBLICLY-FUNDED PRIVATELY FACILITATED ACCESS TO SURVIVAL RESOURCES METHOD as filed on Sep. 29, 2006 and having application Ser. No. 11/537,469;

ELECTRICITY PROVIDING PRIVATELY PROVISIONED SUBSCRIPTION-BASED SURVIVAL SUPPLY UNIT METHOD AND APPARATUS as filed on Oct. 9, 2006 and having application Ser. No. 11/539,798;

PREMIUM-BASED CIVILLY-CATASTROPHIC EVENT THREAT ASSESSMENT as filed on Oct. 9, 2006 and having application Ser. No. 11/539,861; and

PRIVATELY MANAGED ENTERTAINMENT AND RECREATION SUPPLIES PROVISIONING METHOD as filed on Oct. 10, 2006 and having application Ser. No. 11/548,191;

the contents of each of which are fully incorporated herein by this reference.

### TECHNICAL FIELD

This invention relates generally to providing private civil security benefits.

### BACKGROUND

Many citizens of the world have long passed the point when a ready availability of the basic necessities of life is satisfactory in and of itself. Today's consumer-oriented citizens demand, and often receive, an incredibly diverse and seemingly ever-growing cornucopia of consuming and experiential options. Such riches are typically based, in turn, upon a highly interdependent series of foundational infrastructure elements. Examples of the latter include, but are certainly not limited to:

transportation infrastructure such as roads, bridges, railways, and so forth that facilitate the inexpensive and rapid movement of sometimes perishable goods from source to consumer;

communications infrastructure such as telephones, television, radio, and the Internet that facilitate the inexpensive and rapid sharing of news, advice, information, and entertainment; and

the totality of civil services such as police services, fire fighting services, medical services, and so forth that facilitate a sufficient degree of order and predictability to, in turn, permit the complex series of inter-related interactions that modern society requires in order to operate.

As powerful as the machinery of modern life appears, however, modern citizens are today perhaps more at risk of experiencing a serious disruption in their ability to prosper or even to survive en mass than is generally perceived. Providing the necessities of life in general requires a lot of things to all operate, more or less, correctly. To put it another way, a serious disruption to any significant element of civilized infrastructure can produce catastrophic results for a broad swatch of a given civil community. Any number of natural and/or non-naturally-caused events can sufficiently disrupt society's infrastructure and ability to provide one or more life-sustaining resources such as hydration, nutrition, shelter, and the like.

Many people believe and trust that their government (local, regional, and/or national) will provide for them in the event of such a civilly-catastrophic event. And, indeed, in the long view such is clearly a legitimate responsibility owed by any government to its citizens. That such is a consummation devoutly to be wished, however, does not necessarily make it so. Hurricane Katrina provided some insight into just how unprepared a series of tiered modern governmental entities may actually be to respond to even basic survival needs when a civilly-catastrophic event occurs.

Such insights, of course, are not particularly new. Civil preparedness shortcomings occasionally attract public attention and niche marketing opportunities exist with respect to provisioning the needs of so-called survivalists. Indeed, there are those who spend a considerable amount of their time and monetary resources attempting to ready themselves to personally survive a civilly-catastrophic event. Therein, however, lies something of a conundrum.

On the one hand, modern governments typically do little to proactively ensure the bulk survival (let alone the comfort) of their citizens in the face of most civilly-catastrophic events. On the other hand, attempting to take responsible actions to reasonably ensure one's own safety and security can become, in and of itself, nearly a full-time avocation and leave little time to actually enjoy the conveniences and opportunities of modern life. Such individual actions may even be frowned upon by the greater part of society which has grown accustomed and falsely secure with existing efficient just-in-time delivery systems that provide the illusion of plenty while undercutting the perception of risk.

As a result, many (if not most) individuals and their families are largely bereft of access to survival resources that they will need should a civilly-catastrophic event befall them. This shortcoming tends to be relatively comprehensive; most people have ready access to neither a sufficient selection of survival supplies nor a sufficient quantity. For people who do have a store of supplies set aside against such an eventuality, it can be a considerable burden to maintain and ensure the freshness, vitality, and usability of those supplies. At the same time, the same civilly-catastrophic event that occasions their need for supplies will also likely disrupt relevant supply chains enough to cause a partial or complete shortage of supplies at their local merchants. The unfortunate net result is a relatively near term severe need for a variety of survival supplies that will often go unmet for lengthy periods of time.

Such persons are also largely without many options when faced with civilly-catastrophic conditions that create a concurrent need for shelter, evacuation, and/or rescue. For example, in many cases, the only available shelter comprises a public facility such as a school, auditorium, or sports venue on the one hand or ad hoc accommodations obtained at a hotel or via a charity or one's own social network. In some cases, such options may be adequate. In many other cases, however, such options quickly prove inadequate or even dangerous in and of themselves.

### BRIEF DESCRIPTION OF THE DRAWINGS

The above needs are at least partially met through provision of the method to facilitate providing access to a plurality of private civil security resources described in the following detailed description, particularly when studied in conjunction with the drawings, wherein:

FIG. 1 comprises a flow diagram as configured in accordance with various embodiments of the invention;

FIG. 2 comprises a schematic block diagram view as configured in accordance with various embodiments of the invention;

FIG. 3 comprises a schematic block diagram as configured in accordance with various embodiments of the invention;

FIG. 4 comprises a schematic block diagram as configured in accordance with various embodiments of the invention; and

FIG. 5 comprises a schematic block diagram as configured in accordance with various embodiments of the invention.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION

Generally speaking, pursuant to these various embodiments, a plurality of private civil security resources are provided. These private civil security resources comprise, at least in part, civil security provisions, civil security facilities, and civil security services. Authorized beneficiaries are then provided with consideration-based access to this plurality of private civil security resources.

The private civil security provisions can comprise various life-sustaining resources as pertain to various categories of life's necessities, including, for example, breathable air, water, food, and protective clothing. Deployable shelters can also be included if desired. Non-civil security resources (such as, but not limited to, luxury items) can also be provided if desired to supplement such staples.

The civil security facilities can comprise various facilities as have application with respect to protecting such authorized beneficiaries from the effects (including the aftermath, if any) of a civilly-catastrophic event and other dangerous or unpleasant environmental or external influences and forces. These facilities can include, for example, such things as civil security shelters (including both short term and longer term facilities), transportation rally points, trans-shipment facilities and/or distribution facilities for civil security provisions, medical services facilities, training facilities, and so forth.

The civil security services can similarly comprise any of a wide variety of services as have application with respect to protecting such authorized beneficiaries. Exemplary services include, but are not limited to, civil security information services (including information that is both generally and specifically appropriate for individual ones of the authorized beneficiaries), transportation services, rescue services, and so forth.

So configured, authorized beneficiaries will have concrete, predictable access to various categories of privately supplied civil security resources. These steps are readily facilitated without dependency upon (and perhaps even in spite of) governmental oversight, participation, or control. The particular resources provided can vary with the needs and requirements of the authorized beneficiaries. Importantly, via these teachings individuals can benefit from a greatly increased opportunity to bring a considerably improved measure of security into their lives, knowing that, should a civilly-catastrophic event indeed be visited upon them, they will have extraordinary and reliable access to privately facilitated civil security resources.

These and other benefits may become clearer upon making a thorough review and study of the following detailed description. Referring now to the drawings, and in particular to FIG. 1, a corresponding process 100 provides 101 a plurality of private civil security resources. As used herein, “private” will be understood to refer to the opposite of public ownership or control (as exemplified by governmental or ecclesiastical ownership or control) but can include such things as ownership or control via publicly traded ownership (via, for example, publicly traded stock or the like), ownership or control via a not-for-profit entity, or the like.

The plurality of private civil security resources can vary to some degree with respect to category and kind of resource. By one approach, however, such resources will comprise, at least in part, civil security provisions, civil security facilities, and civil security services. Generally speaking, both the civil security provisions and civil security facilities are physical components rather than virtual components or products of the intellect. For example, the civil security provisions will typically comprise physical support and/or protection of one kind or another. Similarly, the civil security facilities will also typically comprise physical assets such as, but not limited to, physical shelters that provide real, substantive physical protection from physical threats to human life. Additional details in this regard are provided below. Also as described in more detail below, however, the civil security services can comprise either physical, real world services or virtual-content services.

These civil security provisions can comprise, for example, a plurality of life-sustaining resources as pertain to a plurality of differing categories of life's necessities. Referring momentarily to FIG. 2, examples in this regard include, but are not limited to, such elemental staples as water 201 (which can comprise water in a pure or impure state and/or other water-based palatable fluids), food 202 (which can comprise a wide variety of raw to highly processed food, nutritional supplements, and so forth), and protective clothing 203 (where “protective” will be understood to refer to protection against one or more potentially hazardous environmental influences such as temperature extremes, precipitation, high velocity winds, and the like but not to matters of fashion, social comment, or the like).

These civil security provisions 200 can further optionally include such components as breathable air 204 (which may comprise, for example, a fresh air supply, an oxygen supply (including but not limited to oxygen generators such as, but not limited to, chemical oxygen generators that often produce oxygen as part of a corresponding exothermic reaction) that can serve as a source of oxygen to mix with other atmospheric components, and/or personal or area air filters that serve to remove harmful airborne contaminants from the air prior to being inhaled by an authorized beneficiary), deployable shelters 205 (where “deployable” will be understood as referring to portable shelters such as tents, canopies, inflatable structures, and the like), and other items 206 as may be appropriate to meet the needs of a given application setting.

By one approach, these civil security provisions can be so provided in an aggregated form. Such might be the case, for example, when accumulating such material at a warehouse or the like. These teachings will also accommodate, however, forming such civil security provisions into corresponding units of civil security provisions. To illustrate, one such unit of civil security provisions as intended, for example, for a single authorized beneficiary might comprise, at least in part, consumable fluids, food, medical supplies, personal hygiene supplies, and environmental threat abatement supplies (such as, for example, breathing masks, hazardous materials handling garb, and the like).

Such a unit of civil security provisions might comprise, for example, a short term unit that comprises a quantity and variety of civil security supplies that are adequate to sustain life for that one authorized beneficiary for, say, at least three days and no more than, say, about fifteen days. As another illustrative example, such a unit of civil security provisions might comprise a longer term unit that comprises a quantity and variety of civil security supplies that are adequate to sustain life for that authorized beneficiary for, say, at least about thirty days but no more than, say, about one year.

Those skilled in the art will recognize and understand that there are various ways by which such adequacy can be determined, measured, and/or established. For example, by one approach, the adequacy of any food contents can be determined as a function of a particular target caloric intake per person on a per day basis. Similarly, the adequacy of any consumable fluid contents could be determined as a function of a particular target quantity intake per person on a per day basis.

The particular time frames suggested in the above examples are intended only to serve an illustrative purpose and are not to be construed as limitations with respect to the practice of these teachings. Those skilled in the art will also appreciate and understand that such a unit of civil security provisions can comprise a quantity of supplies that are intended to support more than one authorized beneficiary for the given period(s) of time. For example, such a unit might comprise a family unit that contains sufficient contents to adequately sustain life for a family of four authorized beneficiaries.

These teachings will also accommodate providing civil security provisions that differ from one another in ways other than by category or kind. For example, both branded and generic versions (or other differentiators with respect to quality) of a same kind of item can be selectively provided if desired.

Referring again to FIG. 1, the civil security facilities will typically comprise physical assets that tend, categorically, to comprise part of a private civil security infrastructure. Such civil security facilities can therefore comprise, for example:

one or more civil security shelters;

one or more rally points (at which authorized beneficiaries can gather in response to a civilly-catastrophic event in order to receive certain civil security services such as transportation to another civil security facility such as a shelter or at least to a location that is away from a location that presently (or imminently) lacks civil security (due, for example, to a civilly-catastrophic event, its aftermath, or both));

a location where at least some of the authorized beneficiary's civil security provisions are available (such as a metropolitan, local, regional, and/or national distribution facility);

a trans-shipment facility for at least some of the civil security provisions (where, for example, dis-aggregated provisions are brought and then parsed and aggregated into corresponding units of provisions destined for particular recipient authorized beneficiaries);

a medical services facility;

and so forth, to note but a few relevant examples in this regard.

These teachings will readily accommodate a wide variety of civil security shelters. For example, this process 100 can provide for both temporary civil security shelters and longer term civil security shelters. A temporary civil security shelter will be understood to comprise a shelter that is configured and arranged to adequately shelter and sustain human life for a relatively short period of time. For example, such a temporary shelter might prove sufficient in this regard for, say, no more than about ten inhabitants for no more than about one week, ten days, 15 days, and the like. Generally speaking, a temporary shelter such as this is designed and intended to only serve as a temporary refuge from the effects of a civilly-catastrophic event. Should longer term accommodations be necessary in a given instance, the inhabitants of such a temporary shelter will often be better served to move to a longer term civil security shelter.

A long term civil security shelter will be understood to comprise a shelter that is configured and arranged to adequately shelter and sustain human life for a relatively longer period of time. For example, such a temporary shelter might prove sufficient in this regard for, say, no more than about 100 inhabitants for up to about three months, six months, one year, or the like. (Again, such numeric examples serve an illustrative, rather than a limiting, purpose only.)

These teachings will also readily accommodate the provision of a longer term civil security shelter up to and including an indeterminate term civil security shelter that is configured and arranged to adequately shelter and sustain human life for an indeterminately long period of time. A shelter of this type will typically be characterized not only by an ability to protect its inhabitants against the rigors of the local environment and to contain a relatively large and varied supply of provisions, but by an ability to exist and operate in a fully or at least substantially independent manner. This can relate in particular to an ability to create and/or otherwise replace its consumable provisions (by, for example, growing food, creating power, and so forth).

It is also possible for such shelters to differ from one another in other ways. For example, such shelters may differ with respect to the non-survival related amenities that are provided. These differences can relate to categories of amenities (such as educational facilities, workshop facilities, food preparation facilities, communications facilities, entertainment and/or recreational facilities, or the like) and/or to a relative level or perception of quality, comfort, convenience, or the like.

The aforementioned civil security services can comprise any of a relatively wide variety of offerings. Some examples include, but are certainly not limited to:
transportation services (where such transportation services are not ordinary mass or individual transit services but instead represent, for example, services that are designed, scheduled, and deployed to effect transportation of dispossessed persons away from a location that substantially lacks civil security (such as an area afflicted by a civilly-catastrophic event) and/or transportation to the aforementioned civil security facilities);
civil security information (including both civil security information that is generally appropriate for a large number (or all) of the authorized beneficiaries (such as civil security preparedness training or the like) as well as information that is specifically appropriate for individual ones of the authorized beneficiaries (such as personal contingency instructions that are formed and provided to guide specific authorized beneficiaries during a time of need regarding particular actions and behaviors that such specific individuals should take and observe when responding to a civilly-catastrophic event as well as consulting services that assist such individuals with respect to fortifying their relative positions and/or otherwise better protecting themselves against the effects of various civilly-catastrophic events);
communications services, including long distance communication services (including, for example, wireless-based services) that are configured and arranged to provide substantially persistent communication services notwithstanding interaction of the long distance communication service with the effects of a civilly-catastrophic event (which may include, for example, satellite-based communications, quickly deployed wireless base stations (that comprise, for example, mobile terrestrial platforms, aerial platforms, and so forth), and the like);
civil security rescue services to facilitate, for example, retrieving selected authorized beneficiaries from dangerous circumstances that are owing, at least in part, to a substantial lack of civil security (including, for example, immediate threats posed by life-threatening influences such as fire, collapsed buildings, lawless unpoliced individuals, and the like as well as less-immediate threats (such as starvation, dehydration, infection, or the like) that can follow a substantial failure of civil infrastructure;
civil security delivery services to provide for deliveries to authorized beneficiaries or the like of such things as their corresponding civil security provisions, fuel, and so forth;
civil security medical services (including but not limited to both fixed location and mobile services;
civil security physical security services (to provide, for example, physical security (such as guards, fences and other barriers, surveillance equipment, and so forth) for residences, businesses, and other physical property of interest to an authorized beneficiary);
independent utilities facilities installation services (to provide, for example, for the installation of fuel-burning generators, renewable energy-based power generators, propane-based heating plants, and so forth at, for example, the residences and businesses of authorized beneficiaries);
post-civilly-catastrophic event social relationship facilitation services (to facilitate rebuilding social networks, friendships, partnerships, and romantic relationships for the survivors of civilly-catastrophic events);
   and so forth, to note but a few relevant examples.

By one approach, and referring momentarily to FIG. 3, this process will readily accommodate providing such civil security provisions 200, civil security facilities 302, and civil security services 303 by a single provider 301. Such a provider 301 can be integrally involved with designing, providing, maintaining, and offering such civil security resources or can comprise an aggregator of such resources, in whole or in part, as are provided by other sources.

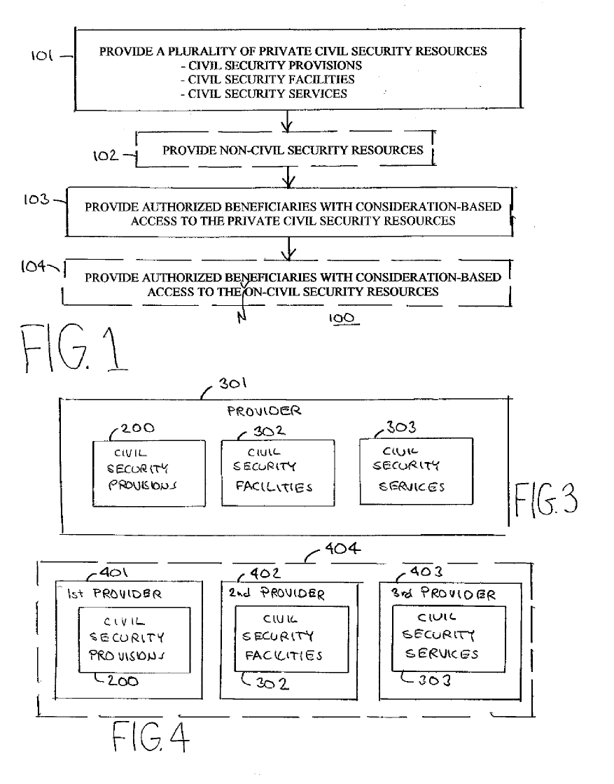

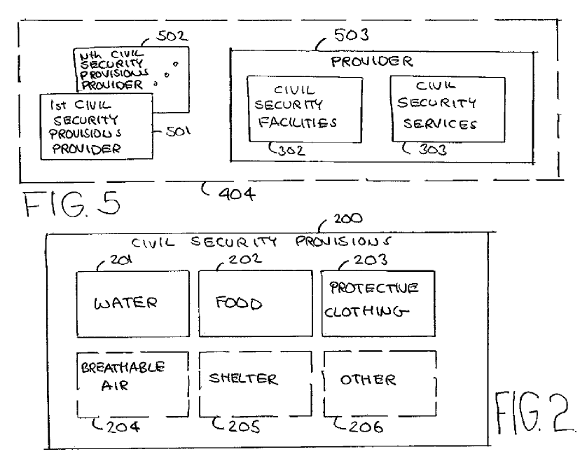

As noted, these teachings can be flexibly applied in various ways in this regard. For example, and referring momentarily to FIG. 4, the civil security provisions 200 can be provided by a first provider 401, the civil security facilities 302 can be provided by a second provider 402, and the civil security services 303 can be provided by a third provider 403 wherein the first, second, and third providers comprise separate entities (as when, for example, these providers comprise separate legal entities having no co-ownership or common points of control). In such a case, these separate providers can meet the needs of this step by acting in an aggregated manner 404 (directly or indirectly) with one another.

Yet other arrangements are possible. For example, and referring momentarily to FIG. 5, a single provider 503 can provide the civil security facilities 302 and the civil security services 303 while the civil security provisions are provided by a plurality of civil security provisions providers represented here by a first civil security provisions provider 501 through an Nth civil security provisions provider 502 (where “N” will be understood to comprise an integer greater than one). Persons skilled in these arts will understand and appreciate that numerous permutations and combinations of these elements as well as other possibilities are available. Such alternatives are well within the scope of these teachings and are rightly considered to comprise a part of this invention.

Referring again to FIG. 1, this process 100 will also optionally provide for the provision 102 of non-civil security resources. Such non-civil security resources comprise supplies and items that are not, strictly speaking, necessary to ensure human survival and hence may be viewed as luxuries. Such items can quite literally span the full gamut of available offerings and can include both unique items (such as original works of art) and more commonly available offerings (such as jewelry, pleasure reading materials, audio playback devices, and so forth). Again, differentiation can exist with respect to actual or perceived levels of quality as pertain to such items if desired.

These steps of providing such civil security resources can further comprise maintaining such resources. This can comprise, for example, maintaining the freshness and usability of civil security provisions (by replacing outdated or soon-to-be outdated supplies), replacing an existing resource with a better substitute that becomes available, maintaining present and/or imminent usability of a civil security shelter or transport vehicle, updating civil security-related information to reflect current data and intelligence, vetting and re-vetting predetermined evacuation and/or transportation routes, educating and training corresponding personnel, and so forth. Such maintenance can be provided, in whole or in part, by the provider of such civil security resources or can be outsourced if desired.

This process 100 then provides for providing 103 authorized beneficiaries with consideration-based access to the plurality of private civil security resources. There are various ways by which such authorized beneficiaries can be identified. For example, by one approach, such authorized beneficiaries can attain this status as per the terms and conditions of a consideration-based private civil security benefits subscription. For example, a given subscription can provide such status for a stipulated one-year period of time for one or more authorized beneficiaries as correspond to that subscription.

By one approach, these subscriptions may be accepted by, for example, a for-profit business. By another approach a not-for-profit business (such as a membership-based-entity) may be the appropriate entity to offer and accept such subscriptions. As used herein, the term “subscription” shall be understood to refer to and encompass a variety of legal mechanisms. Some relevant examples include, but these teachings are not limited to, subscription mechanisms such as:

time-limited rights of access (as where a subscription provides access rights for a specific period of time, such as one year, in exchange for a corresponding series of payments);

event-limited rights of access (as where a subscription provides access rights during the life of a given subscriber based upon an up-front payment in full and where those access rights terminate upon the death of the subscriber or where, for example, a company purchases a subscription for a key employee and those corresponding rights of access terminate when and if that key employee leaves the employment of that company);

inheritable rights of access (as may occur when the subscription, by its own terms and conditions, provides a right of access that extend past the death of a named subscription beneficiary and further provides for testate and/or intestate transfer to an heir);

rights of access predicated upon a series of periodic payments (as where a subscription provides access rights during, for example, predetermined periods of time on a periodic basis as where a subscriber offers month-by-month payments to gain corresponding month-by-month access rights);

rights of access predicated upon a one-time payment (as may occur when a subscriber makes a single payment to obtain a time-based or event-based duration of access rights or, if desired, when a single payment serves to acquire a one-time-only right of access or a perpetual right of access that may be retained, transferred, inherited, or the like);

ownership-based rights of access (as may occur when the subscription provides for ownership rights with respect to the civil security resources, when the subscription is based upon shareholder-based ownership of the provider of such civil security resources, or the like);

non-transferable rights of access (as may occur when the subscription, by its terms and conditions, prohibits transfer of the right of access to the civil security resources from a first named beneficiary to another);

transferable rights of access (as may occur when the subscription, by its terms and conditions, permits conditional or unconditional transfer of the right of access from a first named beneficiary to another);

membership-based rights of access (as may occur when the subscription, by its terms and conditions, establishes a membership interest with respect to the accorded right of access such as, for example, a club-based membership);

fractionally-based rights of access (as may occur when the subscription, by its terms and conditions, establishes a divided interest by and between multiple subscription beneficiaries with respect to a right to access the civil security resources);

non-ownership based rights of access (as may occur when the subscription, by its terms and conditions, establishes the aforementioned right of access via, for example, a lease, a rental, or borrowing construct);

option-based rights of access (as may occur when the subscription, by its terms and conditions, establishes a right for an authorized beneficiary to later obtain access to some or all such civil security resources upon, for example, paying an additional supplemental amount at that time); and/or

credit-based rights of access (as may occur when a given individual predicates their right to access the civil security resources upon a representation, promise, or other credit-based transaction).

If desired, a plurality of differentiated subscription opportunities can be offered in this regard. This plurality of differentiated subscription opportunities can correspond, for example, to providing access to differing selections and/or quantities of the civil security resources. As but one very simple illustration in this regard, such subscription opportunities can differ from one another at least with respect to cost. This, in turn, provides subscriber choice with respect to selecting a particular subscription that best meets their specific needs and/or budget limitations.

These teachings also readily encompass the notion of a given subscriber providing such a subscription for an authorized beneficiary other than themselves. Such might occur, for example, when one family member procures such a subscription for one or more other family members. Another example would be for a company to subscribe on behalf of named key employees, family members of such key employees, and so forth. Other examples no doubt exist. For example, a bearer certificate (or its legal or functional equivalent, such as a gift card) could serve to identify any individual who produces and bears that certificate as an authorized beneficiary.

By one approach, such access can be provided at the whim and convenience of the authorized beneficiaries. If desired, however, such access can be further conditioned in appropriate ways. For example, for many purposes and certainly as pertains to many of the civil security resources it may by appropriate to ordinarily limit such access to situations where such access is triggered, at least in part, by a civilly-catastrophic event having occurred or being likely imminent. Such access may be predicated, if desired, upon a requirement that the civilly-catastrophic event be one that persists in substantial form for more than a predetermined period of time (such as one hour, one day, one week, and so forth) or that causes at least a predetermined amount or degree of infrastructure impairment or other measurable impact of choice (such as a particular level or degree of harm, interference, or negative impact upon a given minimum number of people). In addition, or in lieu thereof, such access may be predicated, if desired, upon a requirement of a particular level of objectivity or subjectively ascertained likelihood that a particular category or kind of civilly-catastrophic event will occur within a particular period of time.

As used herein, “civilly-catastrophic event” will be understood to refer to an event that substantially and materially disrupts a society's local, regional, and/or national infrastructure and ability to provide in ordinary course for at least one life-sustaining resource. Such a civilly-catastrophic event can include both a precipitating event (which may occur over a relatively compressed period of time or which may draw out over an extended period of time) as well as the resultant aftermath of consequences wherein the precipitating event and/or the resultant aftermath include both the cause of the infrastructure interruption as well as the continuation (or worsening) of that interruption.

A civilly-catastrophic event can be occasioned by any of a wide variety of natural and/or non-naturally-caused disasters. Examples of natural disasters that are potentially capable of initiating a civilly-catastrophic event include, but are not limited to, extreme weather-related events (such as hurricanes, tsunamis, extreme droughts, widespread or unfortunately-targeted tornadoes, extreme hail or rain, and the like, flooding, and so forth), extreme geological events (such as earthquakes, volcanic activity, and so forth), extreme space-based events (such as collisions with comets, large asteroids, and so forth, extreme solar flares, and the like), extreme environmental events (such as widespread uncontrolled fire or the like), and global or regional pandemics, to note but a few.

Examples of non-naturally-caused disasters capable of initiating a civilly-catastrophic event include both unintended events as well as intentional acts of aggression such as war, terrorism, madness, or the like. Examples of non-naturally-caused disasters capable of such potential scale include, but are not limited to, nuclear-related events (including uncontrolled fission or fusion releases, radiation exposure, and so forth), acts of war, the release of deadly or otherwise disruptive biological or chemical agents or creations, exposure to a harmful mutagenic influence, and so forth.

In addition, or in lieu thereof, such access can be further conditioned upon other criteria of interest or concern in a given application setting. Such conditional access can be absolute (as when a given individual may be denied any access to any of the civil security resources) or may be relative (as when a given individual is denied access to certain of the civil security resources while being allowed access to certain other of the civil security resources). As a simple illustrative example in this regard, a given individual may habitually and consistently exhibit highly anti-social behaviors; in such a case, the provider of such civil security resources may be willing to provide such a person with civil security provisions but may be reluctant to permit this person to have access to a long term shelter where a certain amount of social buoyancy and ability to compromise with others may be important to the long term ability of that facility to successfully protect its inhabitants.

A very wide variety of criteria can be potentially considered for such purposes. A few illustrative examples would include, but are certainly not limited to, an authorized beneficiary's qualifications (based upon their experiences, aptitude testing, physical condition and capabilities, or the like), measured intelligence (based upon, for example, a traditional intelligence quotient (IQ) test or the like), psychological test results, behavioral test results, race, nationality, citizenship, ancestry, sexual orientation, gender, age, health, political beliefs and/or agenda, educational background, professional title, financial backing, athletic ability, mechanical ability, skills, and/or relationship to a third party entity with whom reciprocal relations regarding the provision of private civil security resources exist (as when multiple providers of such provisions/facilities/services mutually agree to extend civilly-catastrophic event-based reciprocal access to one another's authorized beneficiaries).

As noted earlier, this process 100 will permit optionally providing 102 non-civil security resources. In such a case, this process 100 will then also provide 104 the authorized beneficiaries with consideration-based access to these non-civil security resources as well.

Notwithstanding the provision of such civil security resources, this step of providing access to such resources can readily comprise providing a plurality of different access opportunities. Such opportunities can differ from one another in any number of ways. For example, these opportunities can differ from one another with respect to the specific resources that are included, the quantity of resources that are included, the duration of time during which the aforementioned access may occur, the conditions under which such access can occur, the manner by which such access occurs (for example, whether civil security provisions are delivered to a given authorized beneficiary (and if so, when, how often, and so forth) or must be picked up by the authorized beneficiary from a given distribution point), and so forth.

As one example in this regard, differing access opportunities could comprise, at least in part, a first access opportunity that provides limited access to no more than a first amount of the civil security resources and a second access opportunity that provides limited access to more than the first amount of the civil security resources. In such a case, the first amount can be specified, if desired, as a function of a predetermined quantity of the civil security resources and/or as a function of a predetermined amount of time during which the civil security resources are accessed. As to the latter example, the second access opportunity could differ from the first access opportunity, at least part, by providing an opportunity to have access to the civil security resources at a time that is temporally subsequent to a time when the first access opportunity provides access to the civil security resources (to thereby provide, for example, for a resupply or replenishment opportunity that supplements an original stock of supplies as might correspond to what is provided pursuant to the first access opportunity).

This can comprise, if desired, additionally offering access opportunities that provide access to less than all three categories of the civil security resources noted. As a very simple illustration in this regard, a first access opportunity could provide limited access to only civil security provisions and civil security services while a second, different access opportunity could provide access to both these civil security resources and civil security facilities as well.

By one approach, for example, standard (or customizable) bundles of specific resources, fashioned in different ways, could comprise these different access opportunities. Again, a simple illustrative example may be useful in this regard (though the reader is cautioned that the provided example is but one example of an indeterminate number of possibilities and is not offered with any suggestion of exhaustion in this regard). Consider, in particular, five different access opportunities that are each generally characterized as follows:
A first access opportunity that provides limited access to only a limited selection of the civil security provisions and a limited selection of the civil security services comprising information-related services;
A second access opportunity that provides limited access to only a limited selection of the civil security provisions and a limited selection of the civil security services comprising both information-related services and transportation services (such as civilly-catastrophic event-based transportation to a civil security provisions distribution facility);
A third access opportunity that provides limited access to only a limited selection of the civil security provisions, a limited selection of the civil security services comprising information-related services and transportation services, and a limited selection of the civil security facilities comprising a civil security rally point (or, in other words, an access opportunity that equals that provided by the second access opportunity plus access to the civil security rally point);
A fourth access opportunity that equals that provided by the third access opportunity plus access to a civil security shelter (or shelters); and
A fifth access opportunity that equals that provided by the fourth access opportunity plus access to civil security rescue services.

If desired, a given provider of these civil security resources can provide five such access opportunities to comprise the primary offerings to be provided to the authorized beneficiaries. Each such offering can of course command a different corresponding level of consideration (such as, for example, a different corresponding payment to be proffered for each).

It would of course also be possible to permit an authorized beneficiary having a given one of these access opportunities to extend the scope of the opportunity in exchange for increased consideration. By one approach, such an extension (or upgrade) could be accommodated by simply requiring payment of a price differential as otherwise distinguishes such opportunities in the first instance. Such an approach might be appropriate, for example, when accommodating such a change well prior to even the threat of any particular civilly-catastrophic event. If desired, however, such an extension (or upgrade) might require a considerably higher supplemental payment when the authorized beneficiary seeks the extension (or upgrade) during or following a given civilly-catastrophic event and/or at a time when a given civilly-catastrophic event is perceived as being an imminent occurrence.

It would also be possible to permit some or all of the resources as comprise such an opportunity to be customized to suit the needs and/or preferences of a given authorized beneficiary. In some cases, such customization might be offered without additional corresponding cost. In other cases, and particularly where items and/or services of higher value are being substituted for existing standard items or services, additional commensurate cost might be required. For example, a given authorized beneficiary might wish to upgrade their unit of civil security provisions to include some foods that are not a part of a standard package of supplies or might wish to upgrade from a basic rescue package to a premier rescue offering that offers, for example, a faster guaranteed maximum response time.

If desired, this process will also optionally accommodate providing a key to authorized beneficiaries to thereby facilitate and permit those authorized beneficiaries to access one or more of the civil security resources during an authorized time of need. Such a key may comprise a physical item (such as, for example, an identification card or a physical lock key) and/or a non-physical item (such as, for example, a code such as a personal identification number, a password, or the like). When the key itself comprises a biometric characteristic as corresponds to the authorized beneficiary (such as, but not limited to, deoxyribonucleic acid (DNA), a retinal pattern, an asperity pattern (such as a fingerprint, a palm print, or the like), a voice print, a characteristic typing pattern, and so forth) this step can comprise (in conjunction with acquiring information, materials, and/or samples from the authorized beneficiary to thereby characterize the biometric of choice) providing instructions to the authorized beneficiary regarding presentation of such a biometric in order to gain the desired access. When the key comprises a code, by one approach this step can comprise providing the code to the authorized beneficiary. By another approach, this step can comprise receiving a code of choice from the authorized beneficiary and then providing confirmation to the authorized beneficiary regarding acceptance of that proffered code.

Those skilled in the art will appreciate the considerably flexibility and scalability that these teachings offer. As an example of such flexibility, by one approach, if desired, a base line membership for a given authorized beneficiary can bear a corresponding base line price and offer access to a corresponding set of civil security resources. These resources might comprise, for example:

Pre-civilly catastrophic event delivery of a short-term supply of survival resources comprising at least nutritional consumables. Hydration supplies can also be provided if desired. This short-term supply can comprise, for example, a sufficient quantity of survival resources to support survival of the corresponding authorized beneficiary for at least a predetermined number of days (such as, for example, a ten day period of time).

Pre-civilly catastrophic event access to civil security services comprising consulting services that provide for a preparedness review and inventory as well as preparation of a customized and personalized personal action plan to guide the authorized beneficiary in the event of a civilly-catastrophic event-based time of need.

A right to acquire additional civil security provisions (either for current delivery or to be held pending a civilly-catastrophic event-based time of need) and/or to access civil security facilities or additional services.

So configured, a given authorized beneficiary can obtain a viable, valuable, useful entree into a private civil security paradigm. A base line offering can be provided for a relatively modest initial cost and, if and as the authorized beneficiary becomes more learned and experienced with respect to civil security issues and concerns, additional supplemental kinds and levels of protection and security can be subsequently acquired as needed.

Those skilled in the art will recognize and appreciate that the above described teachings can be applied and leveraged in a variety of ways to achieve a significant number of tangible and concrete benefits (including but not limited to facilitating a relatively higher degree of personal awareness regarding civil security issues, a relatively higher level of resources availability, and/or a relatively more powerful set of motivations for various parties to adopt behaviors that in turn prompt an increased availability of potentially life-preserving resources for a relatively wide consuming audience than might likely be achieved in the absence of such teachings).

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept. As one example in this regard, the notion of civil security provisions can encompass, if desired, weapons of various kinds. Weapons and their ammunition (including, but not limited to, projectile-based weapons of various kinds) can have an important place in ensuring one's survival under certain conditions as they can be used for protection and can also be employed for hunting.

I claim:

1. A method comprising:
providing a plurality of private civil security resources comprising, at least in part:
civil security provisions;
civil security facilities; and
civil security services;
providing authorized beneficiaries with consideration-based access to the plurality of private civil security resources.

2. The method of claim 1 wherein the consideration-based access comprises at least one of:
a time-limited right of access;
an event-limited right of access;
an inheritable right of access;
a right of access predicated upon a series of periodic payments;
a right of access predicated upon a one-time payment;
an ownership-based right of access;
a non-transferable right of access;
a transferable right of access;
a membership-based right of access;
a fractional ownership-based right of access;
a non-ownership-based right of access;
an option-based right of access;
a credit-based right of access.

3. The method of claim 2 wherein providing authorized beneficiaries with consideration-based access to the plurality of private civil security resources further comprises conditioning the access upon at least one of:
an authorized beneficiary's qualifications;
an authorized beneficiary's measured intelligence;
an authorized beneficiary's psychological test results;
an authorized beneficiary's behavioral test results;
an authorized beneficiary's race;
an authorized beneficiary's nationality;
an authorized beneficiary's citizenship;
an authorized beneficiary's ancestry;
an authorized beneficiary's sexual orientation;
an authorized beneficiary's gender;
an authorized beneficiary's age;
an authorized beneficiary's health;
an authorized beneficiary's political agenda;
an authorized beneficiary's educational background;
an authorized beneficiary's professional title;
an authorized beneficiary's financial backing;
an authorized beneficiary's athletic ability;
an authorized beneficiary's mechanical ability;
an authorized beneficiary's skills;
an authorized beneficiary's relationship to a third party entity with whom reciprocal relations regarding a provision of private civil security resources exist.

4. The method of claim 1 wherein the civil security provisions comprise a plurality of life-sustaining resources as pertain to a plurality of differing categories of life's necessities.

5. The method of claim 4 wherein the plurality of differing categories of life's necessities comprise:
breathable air;
water;
food; and
protective clothing.

6. The method of claim 5 wherein the plurality of differing categories of life's necessities further comprises shelter.

7. The method of claim 1 wherein the civil security facilities comprise a civil security shelter.

8. The method of claim 7 wherein the civil security shelter comprises at least one of:
a temporary civil security shelter configured and arranged to adequately shelter and sustain human life for a relatively short period of time;
a long term civil security shelter configured and arranged to adequately shelter and sustain human life for a relatively long period of time.

9. The method of claim 8 wherein the civil security shelter further comprises an indeterminate term civil security shelter configured and arranged to adequately shelter and sustain human life for an indeterminately long period of time.

10. The method of claim 1 wherein the civil security facilities comprise a rally point at which the authorized beneficiaries can gather in response to a civilly-catastrophic event in order to receive the civil security services.

11. The method of claim 10 wherein the civil security services comprise transportation.

12. The method of claim 11 wherein the transportation comprises at least one of:
transportation away from a location that substantially lacks civil security;
transportation to other of the civil security facilities.

13. The method of claim 12 wherein the other of the civil security facilities comprise at least one of:
another of the rally points;
a civil security shelter;
a location where at least some of the authorized beneficiary's civil security provisions are available;
a trans-shipment facility for at least some of the civil security provisions;
a medical services facility.

14. The method of claim 1 wherein the civil security services comprise:
transportation;
civil security information that is both generally and specifically appropriate for individual ones of the authorized beneficiaries;

15. The method of claim 14 wherein the civil security information comprises contingency instructions to guide the authorized beneficiary during a time of need when responding to a civilly-catastrophic event.

16. The method of claim 15 wherein the contingency instructions comprise personal contingency instructions that are specifically formed and provided to guide, specifically, the authorized beneficiary during a time of need regarding particular actions and behaviors that the authorized beneficiary should take and observe when responding to a civilly-catastrophic event.

17. The method of claim 14 wherein the civil security services further comprise civil security preparedness training.

18. The method of claim 1 wherein the civil security services comprise a long distance communications service that is configured and arranged to provide persistent communication services notwithstanding interaction of the long distance communication service with effects of a civilly-catastrophic event.

19. The method of claim 1 wherein the civil security services comprise a rescue service to retrieve selected beneficiaries from dangerous circumstances owing, at least in part, to a substantial lack of civil security.

20. The method of claim 1 wherein the civil security services comprise an information system that an authorized beneficiary can access to receive substantially real-time advice regarding near-term actions to take with respect to a given civilly-catastrophic event.

21. The method of claim 1 wherein the civil security services comprise at least one of:
delivery of at least a portion of the civil security provisions;
transport of dispossessed persons;
physical security;
medical services;
post-civilly-catastrophic event social relationship facilitation;
delivery of fuel;
installation of independent utilities facilities.

22. The method of claim 1 wherein providing a plurality of private civil security resources comprises providing a plurality of private civil security resources that include, but are not limited to:
civil security provisions comprising:
breathable air;
water;
food; and
protective clothing;
civil security facilities comprising:
a civil security shelter;
a rally point at which the authorized beneficiaries can gather in response to a civilly-catastrophic event in order to receive the civil security services; and
civil security services comprising:
transportation;
civil security information that is both generally and specifically appropriate for individual ones of the authorized beneficiaries;
civil security preparedness training; and
a rescue service to retrieve selected beneficiaries from dangerous circumstances owing, at least in part, to a substantial lack of civil security.

23. The method of claim 1 wherein providing authorized beneficiaries with consideration-based access to the plurality of private civil security resources comprises offering a plurality of different access opportunities.

24. The method of claim 23 wherein the plurality of different access opportunities comprises, at least in part:
a first access opportunity that provides limited access to no more than a first amount of the civil security resources;
a second access opportunity that provides limited access to more than the first amount of the civil security resources.

25. The method of claim 24 wherein the first amount is specified as a function of a predetermined quantity of the civil security resources.

26. The method of claim 24 wherein the first amount is specified as a function of a predetermined amount of time during which the civil security resources are accessed.

27. The method of claim 24 wherein the second access opportunity differs from the first access opportunity, at least part, by providing an opportunity to have access to the civil security resources at a time temporally subsequent to a time when the first access opportunity provides access to the civil security resources.

28. The method of claim 23 wherein the plurality of different access opportunities comprises, at least in part:
a first access opportunity that provides limited access to only the civil security provisions and the civil security services;
a second access opportunity that provides limited access to the civil security provisions, the civil security services, and the civil security facilities.

29. The method of claim 23 wherein the plurality of different access opportunities comprises, at least in part:
a first access opportunity that provides limited access to only:
a limited selection of the civil security provisions;
a limited selection of the civil security services comprising information-related services;
a second access opportunity that provides limited access to only:
a limited selection of the civil security provisions;
a limited selection of the civil security services comprising information-related services and transportation services;
a third access opportunity that provides limited access to only:
a limited selection of the civil security provisions;
a limited selection of the civil security services comprising information-related services and transportation services;
a limited selection of the civil security facilities comprising a civil security rally point;
a fourth access opportunity that provides limited access to only:
a limited selection of the civil security provisions;
a limited selection of the civil security services comprising information-related services and transportation services;
a limited selection of the civil security facilities comprising a civil security rally point and a civil security shelter;
a fifth access opportunity that provides limited access to only:
a limited selection of the civil security provisions;
a limited selection of the civil security services comprising information-related services, transportation services, and rescue services;
a limited selection of the civil security facilities comprising a civil security rally point and a civil security shelter.

30. The method of claim 29 wherein any of the different access opportunities are extendable in exchange for increased consideration.

31. The method of claim 1 wherein access to at least one of the plurality of civil security resources is further based, at least in part, upon a civilly-catastrophic event creating a need for such access on the part of the authorized beneficiary.

32. The method of claim 1 further comprising:
providing non-civil security resources;
providing the authorized beneficiaries with consideration-based access to the non-civil security resources.

33. The method of claim 32 wherein the non-civil security resources comprise, at least in part, luxuries.

34. The method of claim 32 wherein providing the authorized beneficiaries with consideration-based access to the non-civil security resources comprises further conditioning access to the non-civil security resources upon a civilly-catastrophic event creating a need for the authorized beneficiary to access the civil security resources.

35. The method of claim 1 wherein providing a plurality of civil security resources comprising civil security provisions comprises providing the authorized beneficiary with:
a short term unit of civil security provisions prior to an occurrence of a civilly-catastrophic event;
access to a longer term unit of civil security provisions in response to an occurrence of a civilly-catastrophic event.

36. The method of claim 35 wherein the civil security provisions comprise, at least in part, consumable fluid, food, medical supplies, personal hygiene supplies, and environmental threat abatement supplies.

37. The method of claim 36 wherein the short term unit of civil security provisions comprises a quantity and variety of civil security supplies to adequately sustain life for a given number and type of people for at least about three days and no more than about fifteen days.

38. The method of claim 37 wherein the longer term unit of civil security provisions comprises a quantity and variety of civil security supplies to adequately sustain life for a given number and type of people for at least about thirty days and no more than about one year.

39. The method of claim 35 wherein the civil security services comprise, at least in part, delivering the longer term unit of civil security provisions to the authorized beneficiary.

40. The method of claim 1 wherein the civil security provisions comprise, at least in part, a supply of breathable air.

41. The method of claim 40 wherein the supply of breathable air comprises, at least in part, an oxygen generator.
